(19) United States
(12) Patent Application Publication
(10) Pub. No.: US 2013/0331649 A1
(43) Pub. Date: Dec. 12, 2013

(54) MAGNETICALLY MANEUVERABLE IN-VIVO DEVICE

(71) Applicants: Magnetecs Corporation, Inglewood, CA (US); GIVEN IMAGING LTD., Yoqneam (IL)

(72) Inventors: Semion Khait, Tiberias (IL); Zvika Gilad, Haifa (IL); Josh Schachar, Santa Monica, CA (US); Laszlo Farkas, Ojai, CA (US); Bruce Marx, Ojai, CA (US); David Johnson, West Hollywood, CA (US); Shawn Hakim, Northridge, CA (US)

(73) Assignees: Magnetecs Corporation, Inglewood, CA (US); GIVEN IMAGING LTD., Yoqneam (IL)

(21) Appl. No.: 13/966,526

(22) Filed: Aug. 14, 2013

Related U.S. Application Data
(63) Continuation of application No. 13/314,273, filed on Dec. 8, 2011.
(60) Provisional application No. 61/420,937, filed on Dec. 8, 2010.
(60) Provisional application No. 61/491,383, filed on May 31, 2011.

(51) Int. Cl. A61B 1/00

(52) U.S. Cl. CPC A61B 1/00158
USPC 600/109

(57) ABSTRACT

An in-vivo device includes a magnetic steering unit (MSU) to maneuver it by an external electromagnetic field. The MSU may include a permanent magnets assembly to produce a magnetic force for navigating the device. The MSU may include a magnets carrying assembly (MCA) to accommodate the permanent magnet(s). The MCA may be designed to generate eddy currents, in response to AC magnetic field, to apply a repelling force. The in-vivo device may also include a multilayered imaging and sensing printed circuit board (MISP) to capture and transmit images. The MISP may include a sensing coil assembly (SCA) to sense electromagnetic fields to determine a location/orientation/angular position of the in-vivo device. Data representing location/orientation/angular position of the device may be used by a maneuvering system to generate a steering magnetic field to steer the in-vivo device from one location or state to another location or state.

### CROSS REFERENCE TO RELATED APPLICATION

The present application is a continuation of U.S. patent application Ser. No. 13/314,273, entitled “MAGNETICALLY MANEUVERABLE IN-VIVO DEVICE”, filed on Dec. 8, 2011 and published as US Patent Application Publication No. US 2012/0149981, which in turn claims the benefit of prior U.S. Provisional Patent Application No. 61/420,937, entitled “MAGNETICALLY MANEUVERABLE IN-VIVO DEVICE”, filed on Dec. 8, 2010, and U.S. Provisional Patent Application No. 61/491,383, entitled “MAGNETICALLY MANEUVERABLE IN-VIVO DEVICE”, filed on May 31, 2011, all of which are incorporated herein by reference in their entirety.

### FIELD OF THE INVENTION

The present invention generally relates to an in-vivo device and more specifically to a magnets and sensing coils assembly for a maneuverable in-vivo device.

### BACKGROUND OF THE INVENTION

In-vivo measuring systems are known in the art. Some in-vivo devices/systems, which traverse the gastrointestinal (“GI”) system, may include an imaging sensor, or imager, for imaging (e.g., capturing images of) the interior of the GI system. An in-vivo device may include one or more imagers. Other in-vivo devices may alternatively or additionally include a medication container and means for administering medication in the GI system. Other in-vivo devices may include means for performing surgical operations in vivo.

Autonomous in-vivo devices are devices that traverse the GI system by being pushed through the GI system by peristaltic force exerted by the digestive system. Autonomous in-vivo devices may also spasmodically move in the intestinal tract in ‘fits and starts’. Moving a device in vivo by using a peristaltic force has drawbacks. For example, the in-vivo device may get stuck somewhere in the GI system for an unknown period of time; the device may capture images in one direction while a nearby area, which may be clinically more interesting, is not imaged sufficiently or at all.

In addition, due to the length of the intestinal tract (several meters), it takes an in-vivo device several hours to traverse the entire GI system. In order to minimize discomfort to a patient and to allow her/him to have as normal life as possible during that time, the patient is asked to wear a data recorder for recording the images captured in vivo, in order for them to be analyzed at a later stage (e.g., after the in-vivo device is finally pushed out of the GI). When a physician reviews the images, or a selection thereof, s/he cannot be certain that all the clinically interesting, or intended, areas of the GI system were imaged. In general, the shorter the time an in-vivo device stays in the GI system, the better (e.g., to reduce discomfort to the patient).

Due to the anatomically-inhomogeneous nature of the GI system—it has anatomically distinct sections such as the small bowel and the colon—and/or to different susceptibility of its various sections to diseases, indiscriminately handling large number of images and frames by the in-vivo device is oftentimes superfluous. In part, this is because relatively less susceptible areas of the intestinal tract are overly imaged. More susceptible areas of the intestinal tract, on the other hand, may be imaged sparingly. The number of images captured from susceptible areas of the intestinal tract may be smaller than clinically desired. It may often be desirable to examine only one specific part of the GI tract, for example, the small bowel (“SB”), the colon, gastric regions, or the esophagus.

While moving an in-vivo device through the GI is beneficial, there are some drawbacks associated with autonomous in-vivo devices in the GI tract. It would be beneficial to have a full control over such movement, including maneuvering the in-vivo device to a desired location and/or orientation and/or angular position or state in the GI system, and maintaining the location/orientation/angular position or state for as long as required or needed.

### SUMMARY OF THE INVENTION

It would, therefore, be beneficial to be able to provide an in-vivo device that would be controllably maneuverable to a desired location and orientation, for example, in the GI system.

An in-vivo device includes a magnetic steering unit (“MSU”) to facilitate maneuvering of the in-vivo device by an externally generated electromagnetic field. The MSU may include a permanent magnets assembly (“PMA”) for interacting with the magnetic field to thereby produce a propelling magnetic force and/or a repelling magnetic force and/or a rotational force, for steering and rotating the in-vivo device. The PMA may include one permanent magnet, or a set of permanent magnets. A permanent magnet may be a ring, or it may be annular or ring-like shaped. The MSU may also include a magnets carrying assembly (“MCA”) that is designed to hold, accommodate, carry or support the permanent magnet or magnets. The MCA may also be designed such that an electromagnetic field may induce eddy currents on the MCA that are sufficient to generate the required repelling force. That is, the MCA may be designed to generate eddy currents as a result of an applied electromagnetic field.

The in-vivo device may also include a multilayered imaging and sensing printed circuit board (“MISP”). The MISP may include circuitry for capturing images, for example, of the GI system, and for transmitting images to an external data recorder. The MISP may also include a sensing coil assembly (“SCA”) for sensing electromagnetic fields in order to facilitate sensing, or determination, of a current location and/or current orientation and/or angular position or state of the in-vivo device. The SCA, which may be part of the MSU, may include one or more (e.g., two, three, etc.) electromagnetic field sensors (e.g., sensing coils) that may be disposed, for example, on one or more printed circuit boards (PCBs). The SCA may include a magnetic field sensing (“MFS”) section that may have embedded or formed therein some of the electromagnetic field sensing coils; other one or more electromagnetic field sensing coils may be included or formed in other PCB sections that may be structurally separated from the MFS section.

A transmitter transmitting the images, or a separate transmitter that may be mounted, for example, on, or be part of, the MISP or SCA, may transmit data that represents location and/or orientation and/or angular position of the in-vivo device to an external system (e.g., to an external maneuvering system) in order to enable the external system to generate a steering magnetic field to move the in-vivo device from a current location/orientation/angular position to a target (e.g., next required or desired) location/orientation/angular position, or to keep the in-vivo device in a certain or given location and/or orientation and/or angular position for as long as required.

In some embodiments, there may be full or some degree of structural and cylindrical/annular overlapping between the MFS section, when folded to a cylindrical shape, and the PMA. For example, the MFS section and the PMA may overlap fully (100%), or partly (less than 100%, e.g., 60%, 30%, etc.). In another embodiment, there may be no overlapping (0% overlapping) between the MFS section and the PMA.

### BRIEF DESCRIPTION OF THE DRAWINGS

Various exemplary embodiments are illustrated in the accompanying figures with the intent that these examples not be restrictive. It will be appreciated that for simplicity and clarity of the illustration, elements shown in the figures referenced below are not necessarily drawn to scale. Also, where considered appropriate, reference numerals may be repeated among the figures to indicate like, corresponding or analogous elements. Of the accompanying figures:

FIG. 1 is a block diagram of an in-vivo device maneuvering system according to an example embodiment;

FIG. 2 is a block diagram of an in-vivo device according to an example embodiment;

FIG. 3A shows a spread out multilayered imaging and sensing printed circuit board (MISP) according to an example embodiment;

FIG. 3B shows another side of the MISP of FIG. 3B;

FIG. 3C shows a partial in-vivo device with the MISP of FIGS. 3A and 3B cylindrically folded according to an example embodiment;

FIG. 3D shows the in-vivo device of FIG. 3C with an optical head according to an example embodiment;

FIG. 4A is a cross-sectional view of a flat sensing coil according to an example embodiment;

FIG. 4B is a cross-sectional view of a flat sensing coil according to another example embodiment;

FIG. 5 shows five layers of a multilayered sensing coils PCB according to an example embodiment;

FIG. 6A shows three annular permanent magnets for inducing a force for propelling and/or rotating an in-vivo device according to another example embodiment;

FIG. 6B shows two eddy current plates for inducing a force for repelling an in-vivo device according to another example embodiment;

FIG. 7A shows a hollow conductive cylindrical structure for inducing eddy current according to an example embodiment;

FIG. 7B shows an eddy current annular disc according to an example embodiment;

FIG. 7C shows an eddy current disc according to an example embodiment;

FIG. 7D shows a magnets carrying assembly (MCA) according to an example embodiment;

FIG. 7E shows a cross-sectional view of the MCA of FIG. 7D;

FIG. 7F shows the MCA of FIG. 7D with three permanent magnets mounted thereon;

FIG. 7G shows an MCA according to another example embodiment;

FIG. 7H shows an MCA according to yet another example embodiment;

FIG. 8 shows a multilayered imaging and sensing PCB (MISP) according to an example embodiment;

FIG. 9A shows the MISP of FIG. 8 introverted or ingathered according to an example embodiment;

FIG. 9B shows the MISP of FIG. 8 in its folded/introverted state and, in addition, a magnet assembly according to an example embodiment;

FIG. 10A shows a cross-sectional view of an in-vivo device with a magnetic steering unit (MSU) according to an example embodiment;

FIG. 10B shows a general view of the in-vivo device of FIG. 10A, where the SCA wraps a PMA according to an example embodiment;

FIG. 11 shows an example magnetic field generating system for maneuvering an in-vivo device according to an example embodiment;

FIG. 12 illustrates an example vector representation of a magnetic field generated by a maneuvering magnetic field generating system according to an example embodiment;

FIGS. 13A and 13B show different cross-sectional views of an in-vivo device in which the MFS section of the SCA and the PMA do not overlap according to an example embodiment;

FIG. 14 shows a general view of the in-vivo device of FIGS. 13A-13B according to an example embodiment; and

FIGS. 15A and 15B show two perspectives of a spread out multilayered imaging and sensing PCB (MISP) of the in-vivo device of FIGS. 13A, 13B, and 14 according to an example embodiment.

### DETAILED DESCRIPTION OF THE INVENTION

The description that follows provides various details of exemplary embodiments. However, this description is not intended to limit the scope of the claims but instead to explain various principles of the invention and the manner of practicing it.

In general, when an autonomous in-vivo device traverses the GI system, the faster the in-vivo device moves through a particular section of the GI system, the more pictures are required to be transmitted from the in-vivo device per unit of time in order to maintain a reasonable distance between GI sites for which successive pictures are taken. That is, if the in-vivo device is at rest, the pictures capturing rate, or image frames generation and/or transmission rate can be made relatively low without risking losing clinical information, and if it moves along the GI system, the pictures/frames generation/transmission rate should be higher in order to take approximately the same number of pictures per unit length. Therefore, some in-vivo imaging systems use a movement estimator for assessing the movement of in-vivo devices in order to enable the imaging systems to deduce the required image capturing rate. For example, in order not to waste physical space in the in-vivo device on a dedicated movement sensing device (e.g., accelerometer) and on the circuitry required to operate it, images captured by the in-vivo device are used to provide the movement indications. However, having full control over the location, orientation and angular position of an in-vivo device in the GI system renders the above-mentioned, and similar, frame rate changing solutions unnecessary, and, in general, such control has many advantages. By “orientation of the in-vivo device” is meant the spatial direction of the longitudinal axis of the in-vivo device, and changing the angular position or state of the in-vivo device from one angular position or state to another may be obtained by rotating the in-vivo device about its longitudinal axis or about any other axis of the in-vivo device.

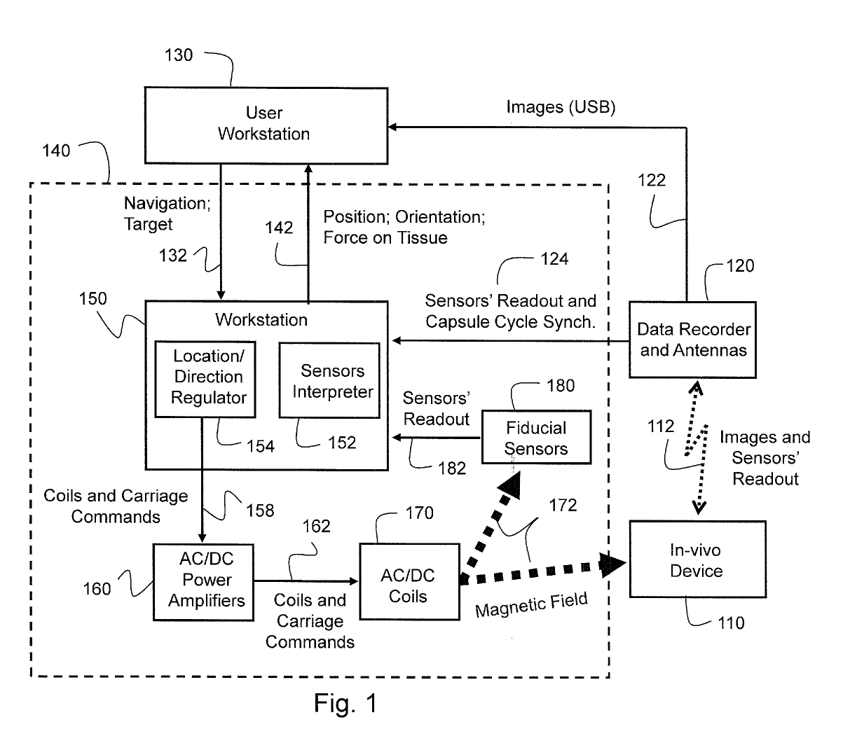

FIG. 1 is a block diagram of a system for magnetically maneuvering an imaging device in vivo, for example for maneuvering an in-vivo imager in the GI system. The system may include a maneuverable in-vivo imaging device 110 for capturing images (i.e., taking pictures) in vivo, and for transmitting the images/pictures; a data recorder and antenna assembly 120 for receiving and processing the images transmitted from in-vivo device 110 and (optionally) for transferring instructions to imaging device 110 (e.g., to change a mode of operation; e.g., to change the images capturing rate), and for transferring the images to a workstation; a user workstation 130 for receiving the images—and optionally, metadata related, for example, to the images—from data recorder 120, and for displaying selected images or a video clip compiled from such images, e.g., to an operator or physician. In-vivo imaging device 110 may include a magnetic steering unit (MSU), which is not shown in FIG. 1, that is capable of sensing three types of magnetic fields: one type of magnetic field for magnetically inducing location and/or orientation and/or angular position signals in imaging device 110, another type of magnetic field for magnetically inducing maneuvering forces for maneuvering imaging device 110, and a third type of magnetic field for externally transferring electrical energy to an energy-picking/harvesting element/circuit in the in-vivo device. Steering of imaging device 110 may be controlled based on the location/orientation/angular position signals.

The system may also include a magnetic maneuvering unit (“MMU”) 140 for generating the magnetic fields that induce the location/orientation/angular position signals in imaging device 110, for interpreting the corresponding location/orientation/angular position data transmitted from imaging device 110, and for generating a magnetic field to steer imaging device 110 to a desired location/orientation/angular position and, if desired or required, for generating the magnetic fields that induce electrical power in imaging device 110.

MMU 140 may include a device displacement module (“DDM”) 150 for translating an intended (e.g., next) location and/or orientation and/or angular position of in-vivo device 110 into a magnetic steering force to position imaging device 110 in the next desired position and/or orientation and/or angular position. MMU 140 may also include AC/DC power amplifiers 160 for generating the electrical signals 162 required to generate the three types of magnetic fields (one for magnetically inducing location and/or orientation and/or angular position signals, the other for generating the steering/rotational force, and the third for transmitting energy). MMU 140 may also include AC coils and DC coils 170 for generating the required magnetic fields from electrical signals 162. MMU 140 may include fiducial electromagnetic sensors 180 for producing an output signal (e.g., current or voltage) that represents or embodies a reference coordinates system relative to which the position and/or orientation of in-vivo device 110 may be sensed, determined, or changed.

Device displacement module (DDM) 150 may include sensors interpreter 152 for interpreting location signals and orientation signals originating from the magnetic steering unit (MSU) of in-vivo imaging device 110 and signals originating from fiducial sensors 180. DDM 150 may also include a location/direction regulator 154 for outputting a regulating signal to AC/DC power amplifiers 160 to generate magnetic fields that correct an ‘error’ in the location, and/or an error in the orientation, of in-vivo device 110. By “error in the location of in-vivo device 110” is meant a difference between a currently sensed location of in-vivo device 110 and a next location of the in-vivo device. By “error in the orientation of in-vivo device 110” is meant a difference between a currently sensed orientation of in-vivo device 110 and a next orientation of the in-vivo device. Data representing or related to the currently sensed location and/or orientation of in-vivo device 100 is shown at 124, and it may be provided to DDM 150, for example from data recorder 120. Data 132 representing or regarding the next location and/or next orientation of the in-vivo device may be provided to DDM 150, for example from a user-operable joystick connected to, or that is part of, user workstation 130.

After in-vivo imaging device 110 is swallowed, or otherwise ingested, it may start capturing images of the GI system, generate an image frame for each captured image, and transmit 112 the image frames to data recorder 120. In order for magnetic maneuvering unit (MMU) 140 to guide and control in-vivo device 110 in the GI system the location and orientation of the device has to be known in real-time. In order to know that, workstation 150 outputs a command 158 to AC/DC power amplifiers 160 to activate/operate coils 170 that generate electromagnetic field 172 to induce electromagnetic signals in device 110 (and, optionally, also in fiducial sensors 180), that indicate, or facilitate sensing of, the current location of in-vivo device 110. The magnetic steering unit (MSU) of in-vivo imaging device 110 may use an on-board sensing coil assembly to sense electromagnetic field 172, and may return a feedback signal, or feedback data, to MMU 140 (e.g., through data recorder 120), as described below. The on-board sensing coil assembly (SCA) of in-vivo device 110 may include three mutually perpendicular, or orthogonal, electromagnetic sensing coils for sensing electromagnetic field 172.

In-vivo device 110 is configured, among other things, to transmit 112 data, which is referred to herein as “location data”, “orientation data”, or “angular position data” (depending on the context) that represent the output signals of the sensing coil assembly (e.g., the sensors' readout), to data recorder 120. In other words, the signals output by the SCA, which may indicate the location and/or orientation and/or angular position of the in-vivo device, may be digitally represented by corresponding data. In one embodiment, in-vivo device 110 may transmit image frames with the location/orientation/angular position data embedded in them, or in selected image frames. In another embodiment in-vivo device 110 may transmit the location/orientation/angular position data independently of the image frames, for example by using a separate or dedicated transmitter and/or a separate communication channel.

Data recorder 120 may relay the location/orientation/angular position data to sensors interpreter 152 of workstation 150. Fiducial sensors 180, which also sense electromagnetic field 172, may be attached to the patient, and/or to a bed on which the patient lies surrounded by coils 170 that generate electromagnetic field 172. The output of fiducial sensors 180 may be also transferred to workstation 150, and location/direction regulator 154 may deduce the location/orientation/angular position of in-vivo device 110 from the location/orientation/angular position data originating from the in-vivo device, for example, relative to a reference coordinates system that may be represented by, or embodied in, the output signal(s) of fiducial sensors 180. Location/direction regulator 154 may also use the data originated from user workstation 130 (e.g., data 132) originated from the in-vivo device to calculate a corrective signal and to output a corresponding command to AC/DC power amplifiers to change electromagnetic field 172 such that in-vivo device 110 would be steered/maneuvered to the intended location and/or orientation. Workstation 150 may transfer various types of data 142 to user workstation 130 for display, etc., for example location data; orientation data; force that the in-vivo imaging device exerts or applies on a tissue wall of the GI system, etc. User workstation 130 may associate images that it receives 122 from data recorder 120, with the various types of data 142.

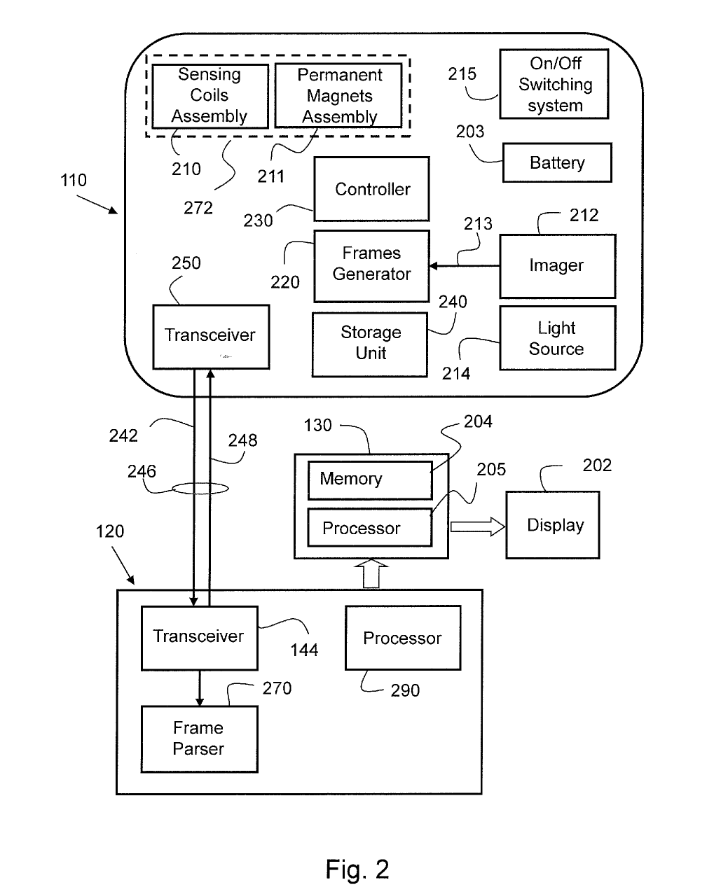

FIG. 2 schematically illustrates an example in-vivo imaging system according to an embodiment. The in-vivo imaging system may include in-vivo imaging device 110, external data recorder 120, workstation 130 (e.g., personal computer), and a display 202. In-vivo imaging device 110 may be, for example, a swallowable device capturing images and transmitting corresponding image frames to an external receiving apparatus, such as data recorder 120. The image frames may be presented in real-time or after processing, be combined into an image stream or video movie for display to a user, for example using display 202.

An in-vivo imaging device may have one or more imagers. By way of example, imaging device 110 include one imager; e.g., imager 212 (numbers of imagers other than one or two may be used, with suitable modifications to the methods discussed herein). In-vivo imaging device 110 also includes a light/illumination source 214, a frame generator 220, a controller 230, a storage unit 240, a transceiver 250, and a power source 203 for powering them. Power source 203 may include a charge storing device (e.g., one or more batteries) with electrical circuit that jointly facilitates transfer of electrical power from an external apparatus to the in-vivo device through electromagnetic induction. Controller 230, among other things, controllably operates illumination source 214 to illuminate areas traversed by in-vivo device 110, and coordinates or schedules the images capturing timing of imager 212. Imaging device 110 may also include a sensing coil assembly (SCA) 210. Controller 230 may coordinate or schedule the reading of the output of sensing coil assembly 210 and temporarily store captured images and related image frames in storage unit 240. Controller 230 may also perform various calculations and store calculation results in storage unit 240.

At the time of or shortly after in-vivo imaging device 110 is swallowed, or after some predetermined delay (e.g., 2 minutes), imager 212 may start capturing images of areas of the GI system. Because natural light does not enter the intestinal tract, imager 212 does not require a light shutter, as opposed to ‘regular’ (i.e., non-swallowable) imagers. The function of the light shutter is, therefore, implemented by the darkness inside the intestinal tract and by intermittently illuminating the FOV of imager 212. Typically, the exposure time of imager 212 is 2-3 milliseconds. Imager 212 includes an image sensor that may be, or include, an array of photo sensor elements (e.g., pixels) such as 256×256, 320×320, 1 Mega pixel or any other suitable array. Imager 212 outputs image data 213 by using a pixel format corresponding to the used pixels. For convenience, pixels are normally arranged in a regular two-dimensional grid/array. By using this kind of arrangement, many common operations can be implemented by uniformly applying the same operation to each pixel independently. Each image data represents a captured image and, optionally, additional selected portions thereof.

Frames generator 220 receives image data 213 and uses the image data to produce an image frame (“frame” for short) for the pertinent captured image. A frame typically includes a header field that contains information and/or metadata related to the frame itself (e.g., information identifying the frame, the serial number of the frame, the time the frame, the bit-wise length of the frame, etc.). A frame may also include an uncompressed version of the image data and/or a compressed version thereof, and a decimated image. The header may also include additional information, for example readout of sensing coil assembly 210 or readout of any additional sensor integrated into device 110.

Controller 230 may operate illumination source 214 to illuminate, for example, four times per second to enable capturing four images per second, and transceiver 250 to concurrently transmit corresponding frames at the same rate. Controller 230 may operate illumination source 214 to capture more images per second, for example seventeen images or more than seventeen images per second, and transceiver 250 to concurrently transmit corresponding frames at the same rate. Controller 230 may operate sensing coil assembly 210 directly or through another (e.g., slave) controller, and write a corresponding sensing data (e.g., the sensing coils readout) into the corresponding frame; e.g., into a frame that is to be transmitted immediately after each sensing of the magnetic field. After frames generator 220 produces a frame for a currently captured image and writes localization data into it, controller 230 wirelessly communicates 242 the frame to data recorder 120 by using transceiver 250. Data recorder 120 may be part of the magnetic maneuvering unit (MMU) 140 or a stand alone unit that is located close enough to the person in order to facilitate receiving and processing of the transmitted frames by data recorder 120.

Data recorder 120 may include a transceiver 244, a frame parser 270, and a processor 290 for managing transceiver 244 and frame parser 270. Data recorder 120 may include additional components (e.g., USB interface, Secure Digital (“SD”) card driver/interface, controllers, etc.), elements or units for communicating with (e.g., transferring frames, data, etc. to) both the regulator 154 of MMU 140 and the processing/displaying system that are configured to process the images captured, and the localization information sensed, by in-vivo device 110, and related data. In one embodiment transceiver 244 receives a frame corresponding to a particular captured image, and frame parser 270 parses the frame to extract the various data entities contained therein (e.g., image data, decimated image associated with, or representing the particular captured image, etc.). In another embodiment, some frames, which are referred to herein as “localization frames”, may be dedicated to carrying or transferring localization data, meaning that such frames may include localization data and, optionally, metadata related to the localization data, but not image data. Using localization frames in addition to image frames that may include both image data and localization data enables reading the localization data (e.g., the output of the sensing coils assembly 210) at a rate that is higher than the images capturing rate. For example, n (n=1, 2, 3, . . . ) localization frames may be transmitted (e.g., by being inserted) between two consecutive image frames, where, in this case, by “image frame” is meant a frame that includes image data and localization data.

The in-vivo imaging system of FIG. 2 may include a workstation 130. Workstation 130 may include a display or be functionally connected to one or more external displays, for example to display 202. Workstation 130 may receive frames (e.g., image frames, localization frames) from data recorder 120 and present them in real-time, for example as live video, or produce a video stream that also contains location and orientation information that may also be displayed on, for example, display 202. Workstation 130 may include a memory, such as memory 204, for storing the frames transferred from data recorder 120, and a processor, such as processor 205, for processing the stored frames. In-vivo imaging device 110 may also include a magnetic steering unit (MSU) 272. MSU 272 may include a sensing coil assembly (SCA) 210 and a permanent magnets assembly (PMA) 211. In-vivo imaging device 110 may also include an “on/off” switching system 215 for switching imaging device 110 on and off.

In some embodiments, data representing the output of sensing coils assembly 210 may be transmitted to data recorder 120 by using image frames, and optionally by using also dedicated frames. The data representing the output of sensing coils assembly (SCA) 210 is (also) referred to herein as “localization data” or “sensing data”. In other embodiments, in-vivo device 110 may use a dedicated narrow-bandwidth telemetry channel to transmit the localization data to data recorder 120. The bit rate of the telemetry channel may be a few hundreds of Kilo bits per second (KBPS) (e.g., between 50 KBPS and 500 KBPS). In order to facilitate the dedicated narrow-bandwidth telemetry channel, transceiver 250 of in-vivo device 110 may include an additional transmitter which is not shown in FIG. 2, and the transceiver 144 of data recorder 120 may include an additional receiver, which is not shown in FIG. 2. In some embodiments, in-vivo device 110 may include two 3-dimensional accelerometers for measuring the direction in which the in-vivo device moves, and the orientation of the in-vivo device.

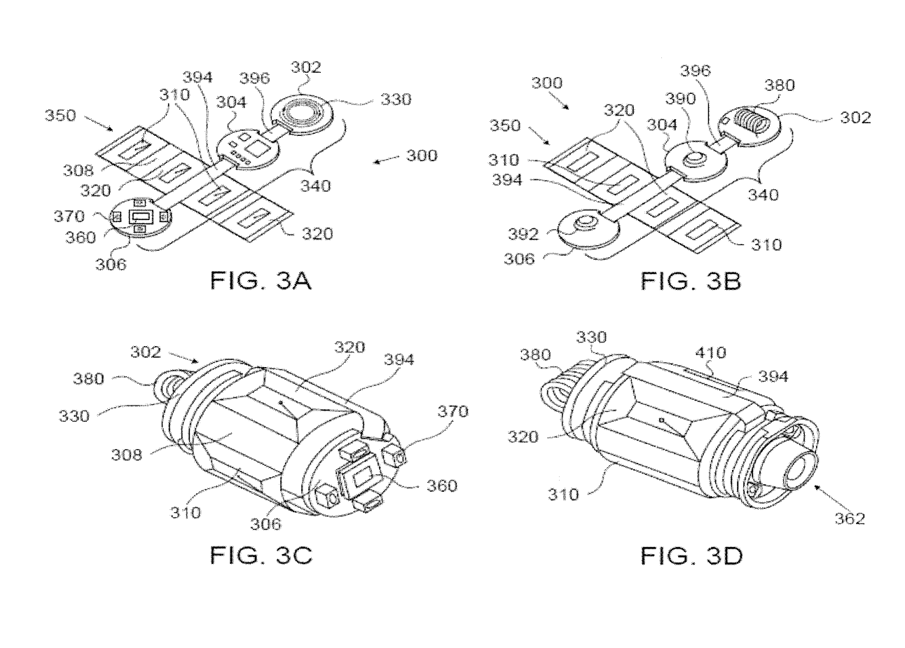

FIGS. 3A through 3B depict a cross-like multilayered imaging and sensing printed circuit board (MISP) 300 of an in-vivo device similar to in-vivo imaging device 110, according to an example embodiment. MISP 300 may be rigid-flex, which means that portions/parts/sections thereof may be rigid whereas other portions, parts or sections thereof may be flexible enough to allow them to be folded into a cylinder-like structure. MISP 300 may be full-flex, which means that all of its portions/parts/sections are flexible. By way of example, MISP 300 is shown including two PCB sections that ‘cross’, or intersect, each other: section 340 and section 350. PCB section 340, which may be rigid-flex, may be regarded as an “imaging section” because it includes the imaging circuitry 306. PCB section 350, which may be fully flexible, may be regarded as a magnetic field sensing (MFS) section because it includes a set of electromagnetic sensing coils for sensing electromagnetic fields by which the current location and/or current orientation and/or current angular position of the in-vivo imaging device may be determined or evaluated. MFS 350 may be part of a sensing coils assembly (SCA) of the MISP 300. The SCA may include one or more additional PCB sections (e.g., PCB section 302) that may include additional electromagnetic field sensing coils (e.g., sensing coil 330).

MISP 300 may include 1-layer portions or sections even though it is generally referred to as a ‘multilayered’ PCB. PCB section 340 may include three rigid sections, designated as 302, 304 and 306, that may be multilayered, and two flexible sections, designated as 394 and 396, that may also be multilayered. Flexible section 394 may connect rigid sections/portions 304 and 306 and be partly sandwiched between layers of these sections/portions. Section 396 may connect rigid sections 302 and 304 and be partly sandwiched between layers of these sections.

Referring to FIG. 3A, an imager 360, which may be similar to imager 212 of imaging device 110, may be mounted on rigid section 306. An illumination source similar to illumination source 214 of in-vivo device 110 may also be mounted on rigid section 306, as shown at 370. By way of example, the illumination source mounted on rigid section 306 includes four light sources which are equidistantly, circle-wise, positioned on rigid section 306. Other electronic components of the in-vivo device (e.g., ASIC, controller, transmitter, crystal oscillator, memory, etc.), may be mounted on section 304 and/or on section 302. An electromagnetic field sensing coil 330 may be mounted on, or be embedded or incorporated into, or formed in PCB rigid section 302. Electromagnetic field sensing coil 330 may functionally be regarded as part, or an extension, of MFS section 350. MFS section 350 and PCB section 302 with electromagnetic field sensing coil 330, thus, form an SCA. In general, an SCA may include, or have disposed thereon, one or more electromagnetic field sensors (e.g., sensing coils, etc.) that may be disposed on one or more PCB sections, and at least one of the one or more PCB sections may be foldable, for example cylindrically or to form a cylinder, while other PCB sections of the SCA may be rigid or partly flexible. At least one of the one or more PCB sections may be foldable to make the electromagnetic field sensors mutually perpendicular. By “partly flexible” is meant flexible but not cylindrically foldable. The other side of sections 302, 304, and 306 may also hold or accommodate additional elements and/or components, as demonstrated in FIG. 3B. Referring to FIG. 3B, section 302 may hold, include, or accommodate an antenna 380 to facilitate radio frequency (RF) communication between the in-vivo imaging device and the data recorder with which the in-vivo imaging device operates.

Sections 304 and 306 may respectively hold, include, or accommodate electrical springs 390 and 392. Section 340 is shown in FIGS. 3A and 3B outspread, but, as part of the in-vivo device assembly process, it is folded such that the rigid sections thereof are stacked in a parallel manner such that rigid sections 304 and 306 can hold, there between, one or more batteries, and the lines normal to the planes of sections 304 and 306 coincide with a longitudinal axis of the in-vivo imaging device. Electrical springs 390 and 392 secure the one or more batteries in place, and electrically connect them to the imaging device's electrical circuit.

Turning again to FIG. 3A, magnetic field sensing (MFS) section 350, which may be part of the SCA, may include electromagnetic sensing coil 310 and electromagnetic sensing coil 320. Electromagnetic sensing coil 310 and electromagnetic sensing coil 320 are shown to be rectangular, but they need not be rectangular. The two sensing coils 310 are collectively referred to as sensing coil 310 because the two sensing coils 310 are electrically, or functionally, interconnected, as shown, for example, in FIG. 5, and thus they form one electrical component (i.e., one sensing coil). Likewise, the two coils 320 are collectively referred to as sensing coil 320 because the two coils 320 may be electrically, or functionally, interconnected, as shown, for example, in FIG. 5, and thus they may form one sensing coil.

Reference numeral 308 designates a flexible multilayered PCB dielectric substrate that holds, includes, or accommodates sensing coils 310 and 320. Each PCB layer of flexible multilayered PCB substrate 308 may hold, include, or accommodate some of the coil turns of sensing coils 310 and/or some of the coil turns of sensing coils 320. Example layers of a flexible multilayered PCB substrate are shown in FIG. 5, which is described below. Magnetic field sensing (MFS) section 350 is shown in FIGS. 3A and 3B outspread, and cylindrically folding it places some turns of sensing coils 310 against other turns of sensing coils 310 such that their normal lines substantially coincide with a same axis (e.g., the ‘X’ axis of the X-Y-Z coordinates system), and some turns of sensing coils 320 against other turns of sensing coils 320 such that their normal lines substantially coincide with another same axis (e.g., the ‘Y’ axis of the X-Y-Z coordinates system). FIG. 3C shows a partly assembled in-vivo imaging device with the folded/introverted multilayered PCB section 340 and the cylindrically folded multilayered MFS section 350. FIG. 3D shows the partly assembled in-vivo device of FIG. 3C with an optical head 362 mounted on top of imager 360 and illuminating source 370.

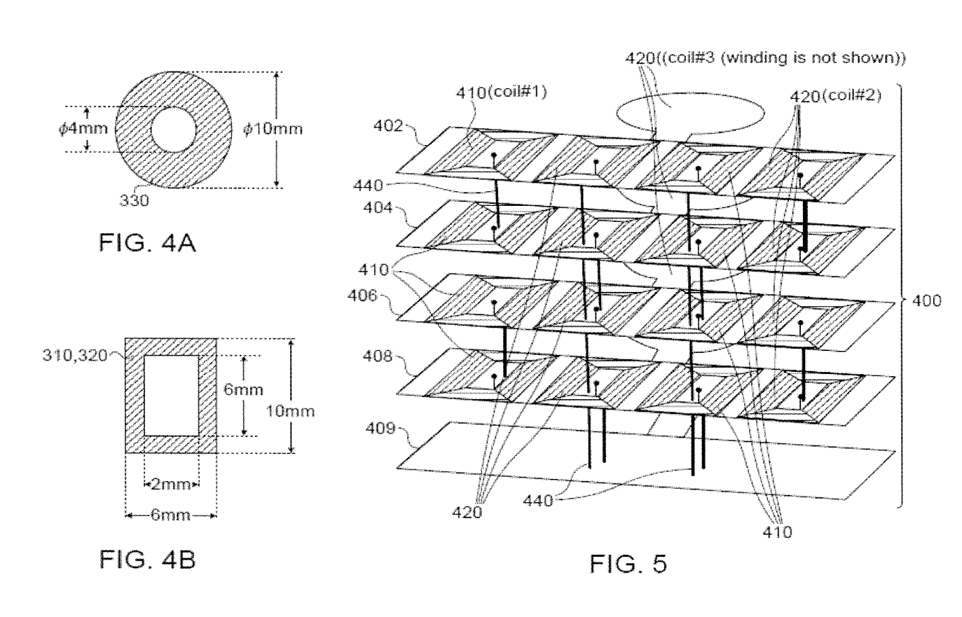

FIG. 4A shows an example cross-sectional area of a sensing coil similar to sensing coil 330 according to an example embodiment. Assume that rigid section 302 of FIG. 3A includes four layers that hold, include, or accommodate the electrical wire/conductors that make up sensing coil 330. Also assume that: the average coil area is 38 mm2; the conductor width is 50 micrometer (μm), and the gap between adjacent conductors is also 50 μm. The overall coil winding, Nt, may, then, be calculated by using formula [1]:

Nt=n×L=30×4=120 [1]

where n is the number of coil turns per layer and L is the number of layers of multilayered rigid section 302.

Also assume that the maximum magnetic field, Bmax, applied to sensing coil 330 is 400 Gauss, and the magnetic field is sinusoidally oscillating at 4 KHz.

The maximum voltage that a sensing coil outputs when placed in a magnetic field may be calculated by using formula [2]:

$$V = \frac{d}{d t} B ( t ) \cdot {A}_{Effective} ( \hat{n} \cdot \hat{B} = 1 ) \qquad [ 2 ]$$

where B(t) is the magnetic field (vector), in Tesla, applied on the sensing coil; A is the coil's area in square meter [m²]; and \hat{n} is the coil direction (it is a unit vector that has no physical units)—i.e., it is a direction normal to the coil's area.

Given the above-mentioned specifics of sensing coil 330 and using formula [2], the theoretical maximum voltage that coil 330 would output is:

|VMAX|=0.04 [Gauss]*2π4,000 [Hz]*1*38*120*10⁻⁶=4.58 [V] [3]

FIG. 4B shows an example cross-sectional area of a sensing coil similar to sensing coils 310, 320 according to an example embodiment. Assume that section 350 of FIG. 3A includes four layers that hold, include, or accommodate the electrical wires/conductors that make up sensing coils 310, 320. Also assume that: the average coil area is 32 mm2 (8 mm×4 mm); the conductor width is 50 micrometer (μm), and the gap between adjacent conductors is also 50 μm. The overall coil winding of each of coils 310 and 320, Nt, may be calculated by using formula [1] above:

Nt=20×4(layers)×2(opposing sides)=160 [4]

Also assume that the maximum magnetic field, Bmax, applied to sensing coils 310, 320 is 400 Gauss, and the magnetic field is sinusoidally oscillating at 4 KHz.

Given the above-mentioned specifics of sensing coils 310 and 320, and using formula [2] above, the theoretical maximum voltage that each of coils 310 and 320 would output is:

|VMAX|=0.04 [Gauss]*2π4,000 [Hz]*1*32*160*10⁻⁶=5.15 [V] [3]

Since section 350, with the coil turns on it, is folded to form a cylindrical structure, a correction factor may be used to compensate for the deviation from the plane of the coil turns. The maximum voltage that each of coils 310 and 320 would output after factoring in the curvature of section 350 is:

|VMAX|=5.15*2\sqrt{2}/π=4.6 [V] [6]

Another factor that reduces the voltage induced in coils 310 and 320, and therefore is to be taken into account, is the eddy current that each coil turn develops as a result of the external AC magnetic. An advantage of the external AC magnetic field is that it induces eddy currents for repelling and restraining the in-vivo device while the device is maneuvered. However, the same AC magnetic field also induces eddy currents in the coils' turns that are harmful because these currents attenuate the voltage induced in the coils' turns. Therefore, equations 3 and 5 are required to be modified to accommodate for the attenuation caused by the eddy current. The attenuation factor was empirically found to be between 2 to 8.

FIG. 5 shows an exploded view of layers of an example multilayered magnetic field sensing (MFS) section 400 according to an example embodiment. By way of example, MFS section 400 includes PCB layers 402, 404, 406, 408, and 409. MFS section 400 hold, include, or accommodates three electromagnetic sensing coils: coil #1 (shown at 410), coil #2 (shown at 420), and coil #3 (at 430 though not shown). PCB layers 402, 404, 406, 408, and 409 are electrically, or functionally, interconnected by using micro vias, which are shown at 440 exaggeratedly long, for clarity. (A “via” is a through-connection electrically connecting between different layers of a printed circuit board.) Layer 409 is a ground/common layer. By using several layers, the overall inductance, and thus the sensitivity, of electromagnetic sensing coils 410 and 420 can be increased, depending, among other things, on the number of coil turns on each layer and on the number of layers holding, including, or accommodating the coil turns.

When the sensing coils assembly (e.g., MFS section 400) is connected to a voltmeter and subjected to a magnetic field, the voltage at the output of the sensing coils assembly can be accurately determined and, there from, the intensity of the magnetic field. Comparison, by the magnetic maneuvering unit (MMU) 140, between the calculated magnetic field and a known map of the magnetic field can be used to calculate the location and orientation of the device. Alternatively, a sensing coils assembly similar to MFS section 400 may be connected to a low impedance device, such as rechargeable batteries or capacitor(s) in order to activate or charge it. An electrical current induced in the sensing coils may be used to charge the batteries or the capacitor and, in doing so, to ‘harvest’ power from external coils 170. Alternatively, a separate coil may circumferentially be disposed on the magnets carrying assembly (MCA) or on one of the permanent magnets that is disposed on the MCA, which is dedicated to picking up energy from an external AC magnetic field.

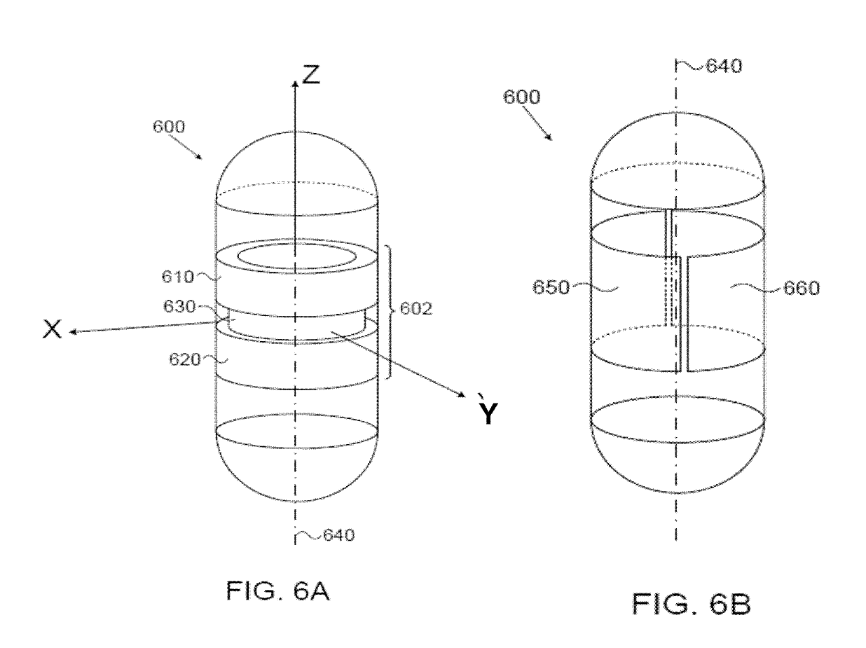

FIG. 6A shows a conceptual permanent magnets setup 602 for steering an in-vivo device 500 in an external DC magnetic field. In-vivo device 500 may be similar to in-vivo device 110 of FIG. 2. Permanent magnets setup 602 may include a permanent magnet PM1, shown at 610, a permanent magnet PM2, shown at 620, and a permanent magnet PM3, shown at 630. Magnets PM1, PM2, and PM3, which are ferrous-conductive elements, may be uniquely magnetized such that in-vivo device 600, a magnetically guided device, is driven by electromagnetic propulsion interaction between external DC magnetic field and permanent magnets PM1, PM2, and PM3.

An external DC magnetic field would force permanent magnets PM1, PM2, and PM3, and therefore in-vivo device 600, to move in a desired direction, for example in the ‘Z’ direction, which may be the direction coinciding with the longitudinal axis 640 of in-vivo device 600, or to apply a torque to rotate in-vivo device 600 to a desired orientation. Variable AC and DC magnetic fields generated externally to the patient (e.g., by magnetic maneuvering unit (MMU) 140) may provide the magnetic forces and rotational torques required to move in-vivo device 600, and to tilt and rotate it within the GI system, based on commands issued by an operator of the magnetic maneuvering system.

Referring to FIG. 6B, an external AC magnetic field system may induce eddy current in ‘eddy-current plates’ 650 and 660 that will result in repulsive forces that moderate, suppress or stabilize the propulsion dynamics resulting from, or associated with, the operation of permanent magnets PM1, PM2, and PM3.

The permanent magnets shown in FIG. 6A and the eddy-current plates shown in FIG. 6B are illustrative. Since the in-vivo device (e.g., in-vivo device 110) has a little space to accommodate the imaging circuit, which includes the imager, transmitter, etc., the permanent magnets, the eddy-current plates, and the sensing coils, the in-vivo device has to be meticulously designed, both mechanically and electrically, in order to enable all the components of the in-vivo device to mechanically coexist in the in-vivo device's housing and to operate without interfering with one another—for example without the RF communication between the in-vivo device and the data recorder affecting the maneuvering magnetic fields and the sensing magnetic fields, and vice versa; and without one type of magnetic field (e.g., the sensing magnetic field) affecting the other type of magnetic field (e.g., maneuvering magnetic field); and without one component (e.g., the permanent magnets) functionally screening or blocking another component (e.g., the sensing coils), etc. Since the imaging section and the MFS section of the magnetic imaging and sensing printed circuit board (MISP) have to be folded into the in-vivo device's housing without entangling with the other components of the in-vivo device, the layout of the MISP and the selection of the components mounted on the MISP are subject to stringent design constraints.

An in-vivo device such as the one disclosed herein may be useful in promoting medical diagnostic procedures or other procedural operations that require or can use in vivo steering of an in-vivo device, for example through the GI system. An in-vivo device (e.g., in-vivo device 600) may be provided with at least two permanent magnetic rings (which are also referred to herein as “permanent annular magnets”), or disks or plates, each of which may have anisotropic magnetic properties.

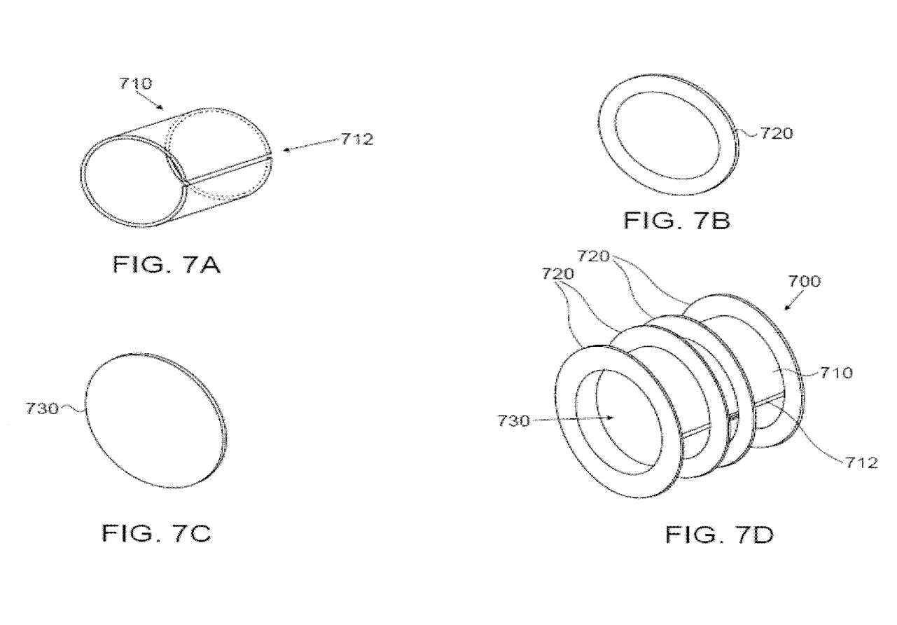

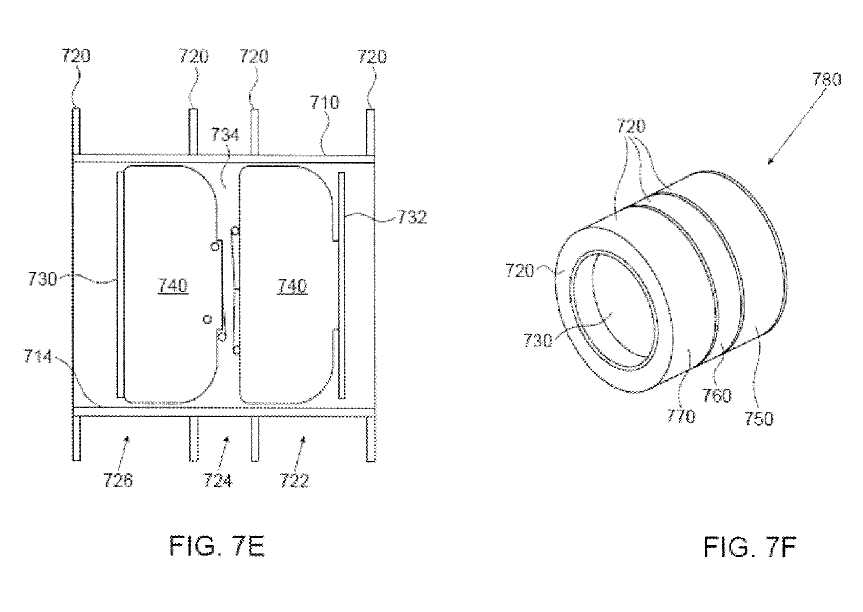

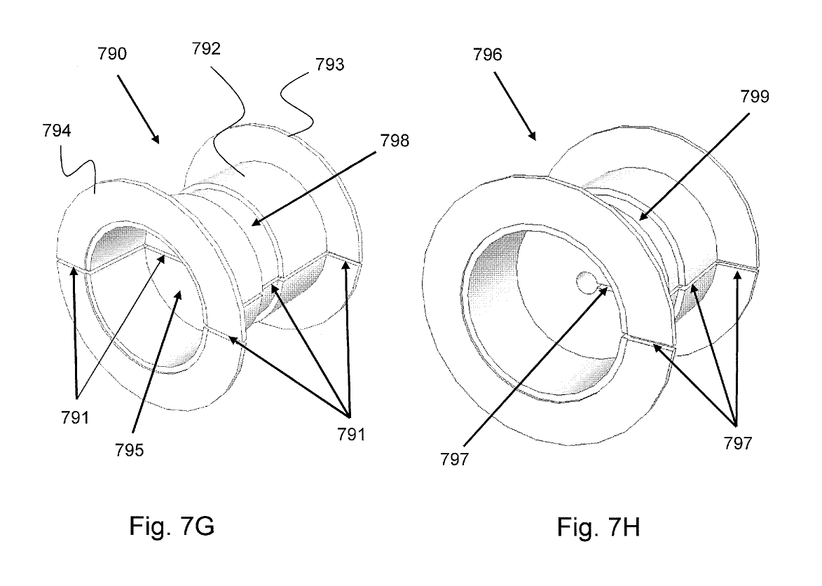

FIGS. 7A, 7B, and 7C respectively show an electrically conductive tubular object 710 for inducing eddy current thereon when tubular object 710 is placed in an AC magnetic field, an electrically conductive annular disc 720, and an electrically conductive disc 730. Conductive electrically conductive tubular object 710, conductive annular discs similar to annular disc 720, and conductive discs similar to conductive disc 730 make up a magnets carrier assembly (MCA) 700, which is shown in FIG. 7D.

When an AC magnetic field is applied to tubular object 710, annular disc 720 and disc 730, eddy currents flow on the surface of these objects. A slit 712 disconnects the electrical continuity of these elements in order to reduce parasitic currents. Without slit 712, the eddy currents induced by the external AC magnetic filed may induce adversary eddy currents that may degrade the efficiency of MCA 700 as it is levitated, or otherwise maneuvered, under the pertinent laws of physics (e.g., Lenz's Law).

More than one slit may be used: FIG. 6B shows two eddy current plates 650 and 660 that are separated by two slits; in other embodiments other slits may be used. The slits setup (e.g., number of slits, their shape and relative location/orientation) may be chosen such that the repulsive force caused by or resulting from the eddy current is optimized. Magnets carrier assembly (MCA) 700 of FIG. 7D is an electrical conductor. MCA 700 may be made entirely of silver, or aluminum, or copper, or any other suitable electrically conducting material. Alternatively, MCA 700 may be made partly of silver, partly of aluminum, etc. For example, tubular object 710 may be made of silver and the other parts of MCA 700 (e.g., conducting annular discs, conducting discs) may be made of aluminum. Alternatively, MCA 700, or parts thereof, may be an electrically conducting alloy.

In general, MCA 700 may serve three purposes: (1) holding or accommodating the (annular, ring or ring like) permanent magnets (e.g., PM1, PM2, PM3 of FIG. 6A) required/used to propel the in-vivo imaging device through the GI system by using a DC magnetic field, (2) facilitating generation of the surface eddy currents that exert a repulsive/restraining/drag forces on the imaging device, and (3) housing the batteries of the in-vivo device. FIG. 7D shows a 3-dimensional view of MCA 700. The design of MCA 700 factors in various mechanical and operational/functional constraints, for example as mentioned above. A cross-sectional view of MCA 700 is shown in FIG. 7E. FIG. 7E also shows two batteries 740 of in-vivo device. FIG. 7F shows a complete magnets assembly 780 that includes MCA 700 of FIG. 7D and three annular permanent magnets 750, 760, and 770 that are mounted on MCA 700.

Turning again to FIG. 7E, by way of example four electrically conductive annular discs 720 are used for augmenting/enhancing the induced eddy current. As shown in FIG. 7E, annular conductive discs 720 are perpendicularly disposed on the peripheral surface of conductive tubular object 710 to circumferentially form, in this example, three open annular channels 722, 724, and 726 on the periphery around conductive tubular object 710. Open annular channels 722, 724, and 726 are used to hold or accommodate permanent annular magnets, or permanent magnetic rings, 750, 760, and 770, respectively, as shown in FIG. 7F. The number of annular open channels may be three, less than three, or more than three. An annular open channel may include one or more permanent magnet. By way of example, each annular open channel in FIG. 7F includes one permanent magnet. Annular conductive discs 720 in FIG. 7E are mutually parallel; in other embodiments the annular conductive discs may be unparallel.

FIG. 7E also shows a first conductive disc 730 and a second conductive disc 732 for further augmenting/enhancing the induced eddy current. Conductive disc 730 is mounted on a first side (e.g., on the left-hand side) of conductive tubular object 710, and conductive disc 732 is mounted on a second side (e.g., on the right-hand side) of conductive tubular object 710. As shown in FIG. 7E, conductive discs 730 and 732 are mounted opposite one another. One or more batteries may be contained in a chamber 734 formed by conductive disc 730, conductive disc 732, and a portion of the inner surface 714 of conductive tubular object 710.

An in-vivo device may be maneuvered by electromagnetic repulsion-levitation interaction between external static and time varying magnetic fields that may be generated, for example, by external AC/DC coils 170, and any of the elements shown in FIG. 7A through FIG. 7F. The elements shown in FIG. 7A through FIG. 7F, or some of these elements, may contain uniquely magnetized ferrous-conductive materials and have anisotropic magnetic properties. These elements (e.g., elements 710, 720, 730, 732) may be made of or include materials such as NdFe and/or other highly-magnetized materials. Referring to FIG. 7F, one or more of the permanent magnets 750, 760, 770 may be magnetized in a direction that is parallel to the longitudinal axis (i.e., in the axial direction) of the in-vivo device (e.g., axis 640, shown in FIG. 6A) and the other permanent magnet(s) may be magnetized in a radial manner in order to produce a (dual) axial-radial perpendicular field around the in-vivo device. The electrically conductive tubular object 710, annular disc 720, and discs 730, 732 may be made, partly or wholly, of Silver or Aluminum to minimize resistive losses. Other super magnetic materials and conductors which provide similar magnetic and electric responses may be used.

FIG. 7G shows an MCA 790 according to another example embodiment. MCA 790 includes a through slit 791 that ‘cuts’ MCA 790 into two symmetrical halves. MCA 790 includes a tubular object 792. By way of example, MCA 790 also includes two annular conducting discs 793 and 794, each annular disc being disposed on one side of tubular object 792, and one disc 795 that is internally disposed in the middle of cylindrical structure 792. FIG. 7H shows an MCA 796 according to yet another example embodiment. MCA 796 is similar to MCA 790, except that MCA 796 has a slit 797 that ‘goes’ only half-way through MCA 796. Reference numerals 798 in FIGS. 7G and 799 in FIG. 7H respectively denote circumferential recesses in tubular objects 790 and 796. Each of circumferential recesses 798 and 799 may hold or accommodate a permanent magnet and, on top of the permanent magnet, an energy-picking coil dedicated to pick up, or harvest, electrical energy through electromagnetic induction. The MCA, or selective elements thereof (e.g., the tubular object) may be slotted in a different way to obtain a desired maneuvering effect.

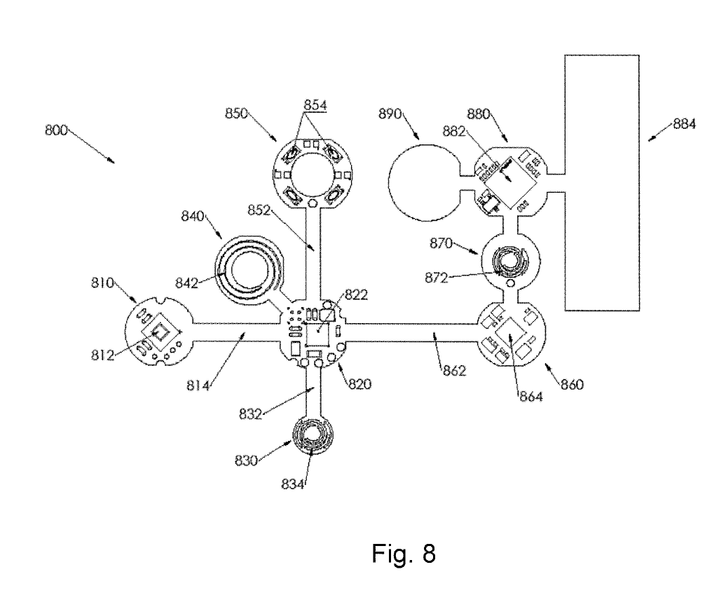

FIG. 8 shows a multilayered imaging and sensing PCB (MISP) 800 according to an example embodiment. Like MISP 300, MISP 800 includes two main parts: (1) an imaging part, and (2) a sensing and energy-picking part. In general, a MISP may include a primary PCB branch, one or more secondary PCB branches that may intersect the primary PCB branch, one or more tertiary PCB branches that may intersect one or more of the secondary PCB branches, etc. By way of example, MISP 800 includes a primary PCB branch, two secondary PCB branches that intersect the primary PCB branch, and a tertiary PCB branch that intersects one of the secondary PCB branches.

The primary PCB branch may include PCB portions 810, 820 and 860, a PCB portion 814 that connects portions 810 and 820, and a PCB portion 862 that connects portions 820 and 860. A first secondary PCB branch may include PCB portions 820, 830, 840 and 850, a PCB portion 832 that connects PCB portions 830 and 820, a PCB portion 852 that connects PCB portions 850 and 820, and, similarly, a PCB portion that connects PCB portions 840 and 820. A second secondary PCB branch may include PCB portions 860, 870, 880, a PCB portion that connects PCB portions 860 and 870, and a PCB portion that connects PCB portions 870 and 880. The tertiary PCB branch includes PCB portions 880, 884, and 890.

Some portions of MISP 800 may be common to two or more PCB branches: PCB portion 820 is common to the primary PCB branch and the left secondary branch; PCB portion 860 is common to the primary PCB branch and the right secondary branch; and PCB portion 880 is common to the right PCB branch and the tertiary branch. The common PCB portions of MISP 800 may be thought of as ‘PCB hubs’, or PCB intersection hubs/points, and the PCB branches of MISP 800 may be regarded as being functionally interconnected via the intersection hubs.

Each PCB portion of MISP 800 may hold, include, or accommodate an optical and/or electrical component of the in-vivo device. For example, PCB portion 810 may hold, include, or accommodate an imager, as shown at 812; PCB portion 820 may hold, include, or accommodate a crystal oscillator, as shown at 822; PCB portion 830 may hold, include, or accommodate a first spring coil, as shown at 834; PCB portion 840 may hold, include, or accommodate an RF communication antenna, as shown at 842; PCB portion 850 may hold, include, or accommodate a light emitted diode (“LED”) ring, as shown at 842 (the LED ring is shown including four LEDs, but it may include less than four LEDs or more than four LEDs); PCB portion 860 may hold, include, or accommodate a switch, as shown at 862; PCB portion 870 may hold, include, or accommodate a second spring coil, as shown at 872; PCB portion 880 may hold, include, or accommodate a microcontroller, as shown at 882; PCB portion 884 may hold, include, or accommodate X-Y sensing coils (the sensing coils are not shown in FIG. 8), for respectively sensing electromagnetic fields in the X axis and in the Y axis; PCB portion 890 may hold, include, or accommodate a Z-axis sensing coil (the sensing coil is not shown in FIG. 8), for sensing an electromagnetic field in the Z axis, where the Z axis may coincide with the longitudinal axis of the in-vivo device.

MISP 800 may be fully flexible or partly rigid and partly flexible (i.e., it may be rigid-flex, meaning that it may include flexible portions and rigid portions). For example, each of MISP portions 810, 820, 830, 840, 850, 860, 870, 880, and 890, may be rigid or flexible. MISP portion 884 may be flexible to enable folding it into a cylindrical shape. Each of the connection portions of MISP 800 may be flexible. Each portion of MISP 800 may have n layers (n=1, 2, 3, . . . , ), and the various circuit components mounted on the various layers may be electrically interconnected through micro vias. MISP 800 is shown contained in housing 888 of the in-vivo imaging device.

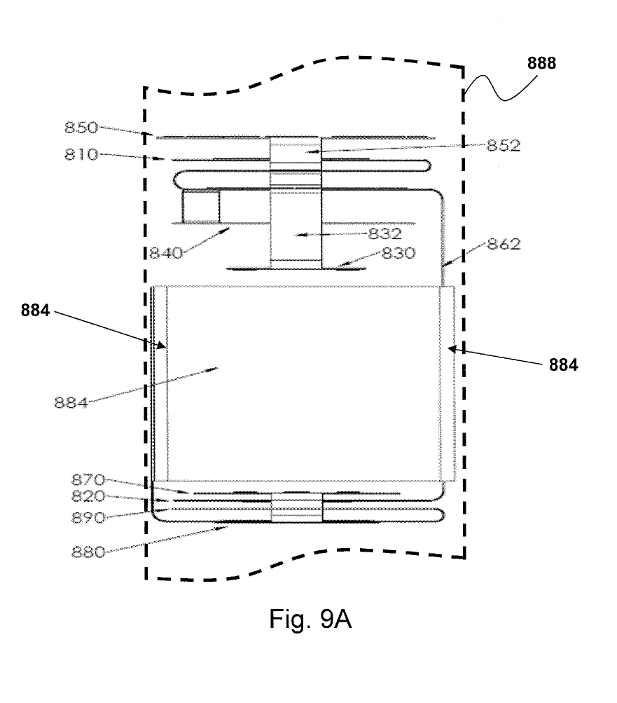

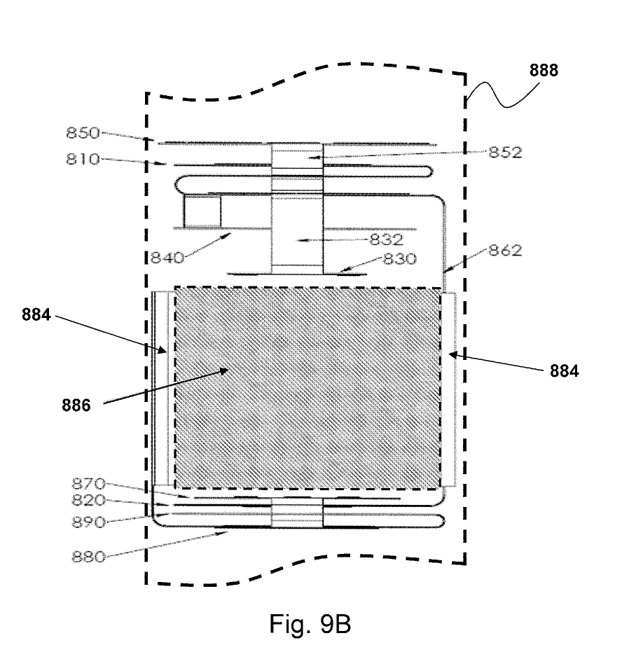

FIG. 9A shows MISP 800 in its folded/introverted state, where like referral numbers represent like PCB section/portions in FIG. 8. FIG. 9B shows MISP 800 in its folded/introverted state and, in addition, a magnet assembly 886 which may be similar to magnet assembly 780 of FIG. 7F. Referring again to FIG. 7D, magnets carrier assembly (MCA) 700 is an electrical conductor. MCA 700 may be made entirely of silver, or aluminum, or copper. Alternatively, MCA 700 may be made partly of silver, partly of aluminum, etc. Alternatively, MCA 700 may be an electrically conducting alloy.

Since magnets carrier assembly (MCA) 700 is made of electrically conducting material(s), it may shield the sensing coils of the MISP and, therefore, degrade its performance. Therefore, as shown in FIG. 9B, magnet assembly 886, as a whole (the magnets with the magnets carrying assembly (MCA)), is snugly fitted to be contained in or generally circumscribed by folded/introverted MISP 800 in order to mitigate mutual interference between them.

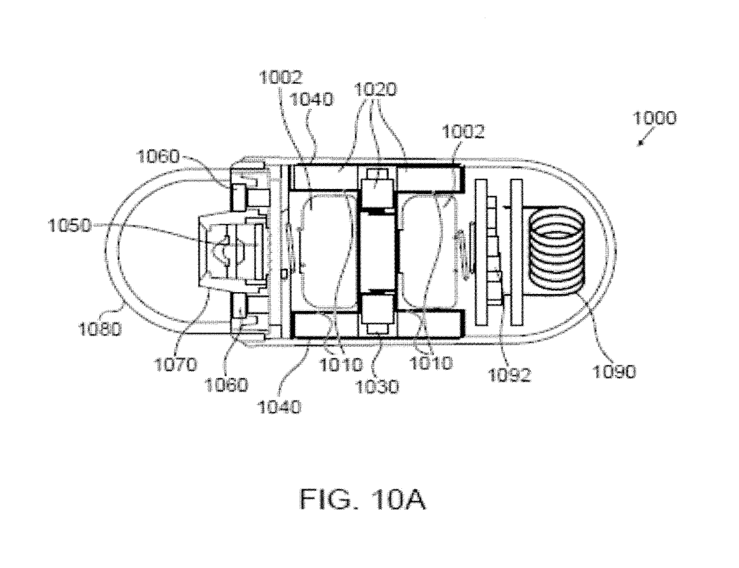

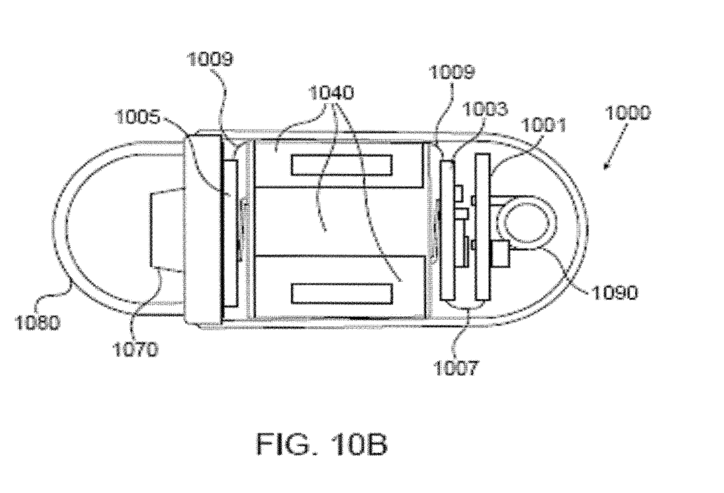

FIG. 10A shows a cross-sectional view of an in-vivo capsule 1000 with a magnetic steering unit (MSU) according to an example embodiment. By way of example, the MSU of in-vivo capsule 1000 includes a magnetic carrier assembly (MCA) 1010; permanent magnets 1020; and magnetic field sensing (MFS) section 1040. Although MCA 1010 looks different from MCA 700 of FIG. 7D, it functions in the same way as, and it may be replaced by, MCA 700 (with the required changes; e.g., replacing the middle permanent magnet with a larger magnet). MFS section 1040 may be identical or similar to MFS section 350 of FIG. 3A. FIG. 10A also shows an energy-picking coil 1030 that may be used to pick up electrical energy from an external AC magnetic field for powering in-vivo capsule 1000.

FIG. 10A also shows an imager 1050, which may be similar to imager 360 of FIG. 3A; an illumination source 1060, which may be similar to illumination source 370 of FIG. 3A; an optical head 1070, which may be similar to optical head 362 of FIG. 3D; an optical window 1080; a communication antenna 1090, which may be similar to communication antenna 380 of FIG. 3B, a transceiver circuit 1092, and batteries 1002.

FIG. 10B shows the in-vivo capsule 1000 of FIG. 10A with a folded multilayered imaging and sensing printed circuit board (MISP) according to an example embodiment. Regarding FIGS. 10A and 10B, like reference numerals refer to like elements/components. The MISP of in-vivo capsule 1000 includes MFS section 1040, which is shown folded; an imaging section that may be similar to imaging section 340 of FIG. 3A. By way of example, the imaging section of in-vivo capsule 1000 includes PCB rigid sections 1001, 1003, and 1005 (which may respectively be similar to rigid sections 302, 304, and 306 of FIG. 3A), and flexible/foldable sections 1007 and 1009 (which may be similar to sections 394 and 396 of FIG. 3A).

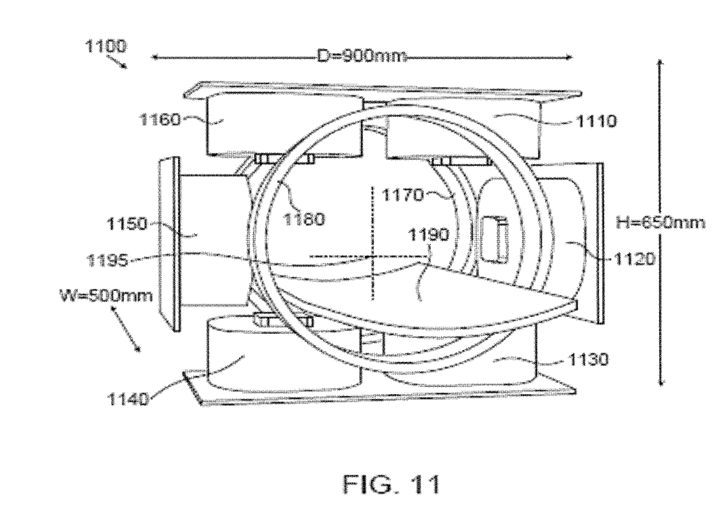

FIG. 11 shows a magnetic maneuvering system 1100 according to an example embodiment. Magnetic maneuvering system 1100 includes a magnetic field generator that includes DC/AC magnetic coils 1110, 1120, 1130, 1140, 1150, 1160, 1170, and 1180 to generate DC and AC magnetic fields to maneuver an in-vivo device swallowed by a patient lying on bed 1190. The DC coils and the AC coils may form a magnetic field within the ‘maneuvering space’ 1195, which resembles the magnetic field shown in FIG. 11.

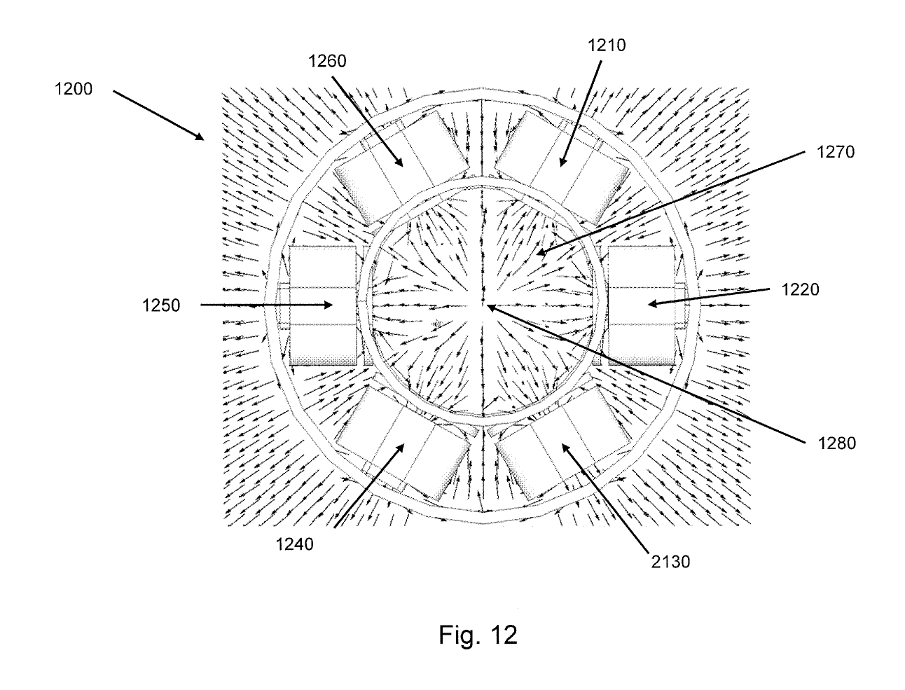

FIG. 12 is an example magnetic vector field generated by magnetic coils 1210, 1220, 1230, 1240, 1250, and 1260. Magnetic vortex 1280 is located at the center of the vector field 1270. Magnetic vortex 1280 is a point, or region, from which field-vectors originate and spread out symmetrically through each of coils 1210 through 1260. The location of magnetic vortex 1280 may be moved, and its shape set, by independently controlling the magnitude and direction of the currents flowing through the coils. Dynamic manipulation of the magnetic vector field changes the characteristics (e.g., location, direction, strength, orientation) of magnetic vortex 1280, and thus it changes the magnetic forces resulting from the interaction between the magnetic fields and the permanent magnets and the eddy-current inducing magnets carrier assembly (e.g., MCA 700), causing the in-vivo imaging device to move as a result of these forces.

One embodiment of the invention includes a swallowable capsule or a swallowable in-vivo device including an MSU maneuverable by an externally generated electromagnetic field. The MSU may include a PMA which interacts with the magnetic field to produce a force such as propelling force and/or a repelling force and/or a rotational force, for maneuvering/steering and/or rotating the in-vivo device. The PMA may include at least one permanent magnet, and an MCA to hold, or accommodate, the at least one permanent magnet, said MCA designed to induce eddy currents as a result of an applied electromagnetic field. The capsule or device may include an SCA for sensing electromagnetic fields in order to facilitate sensing of a current location and/or current orientation and/or current angular position of the in-vivo device. The SCA may include electromagnetic field sensing coils, for example disposed on one or more foldable printed circuit boards sections.

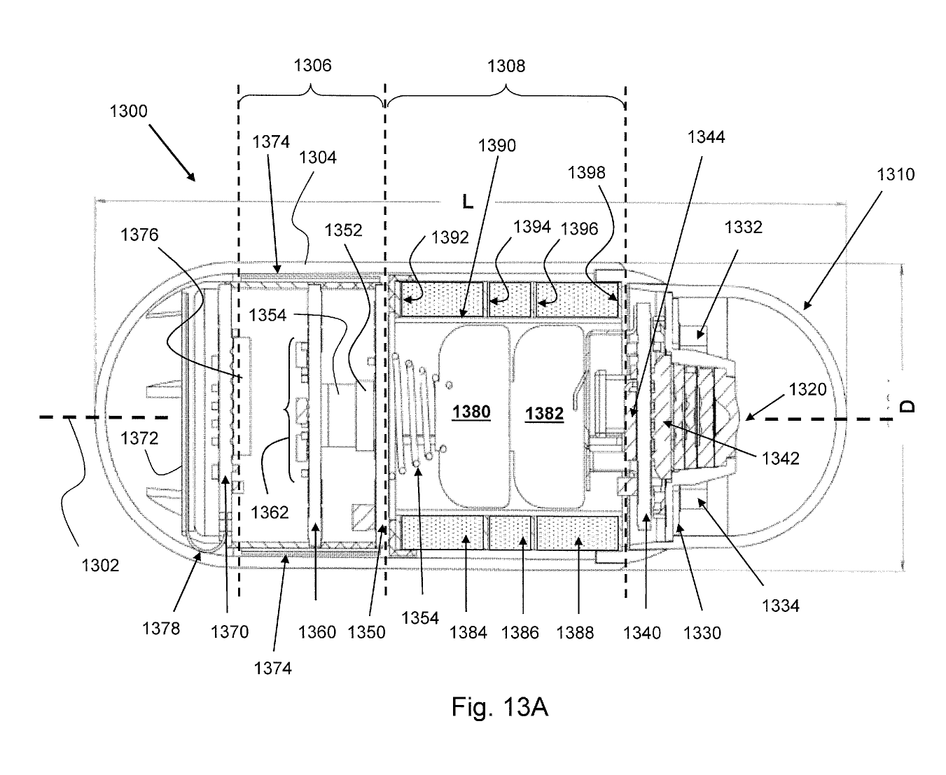

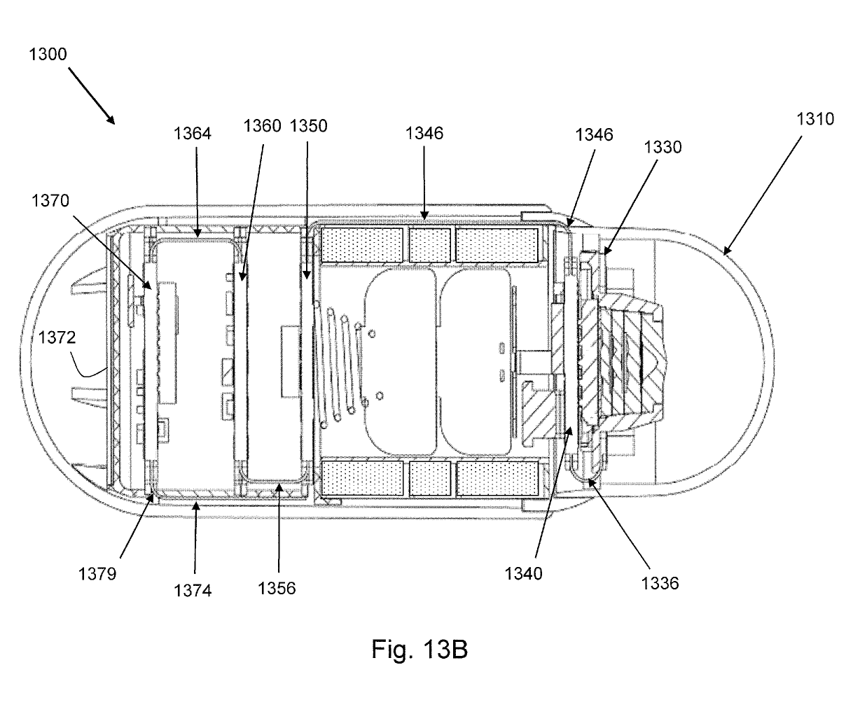

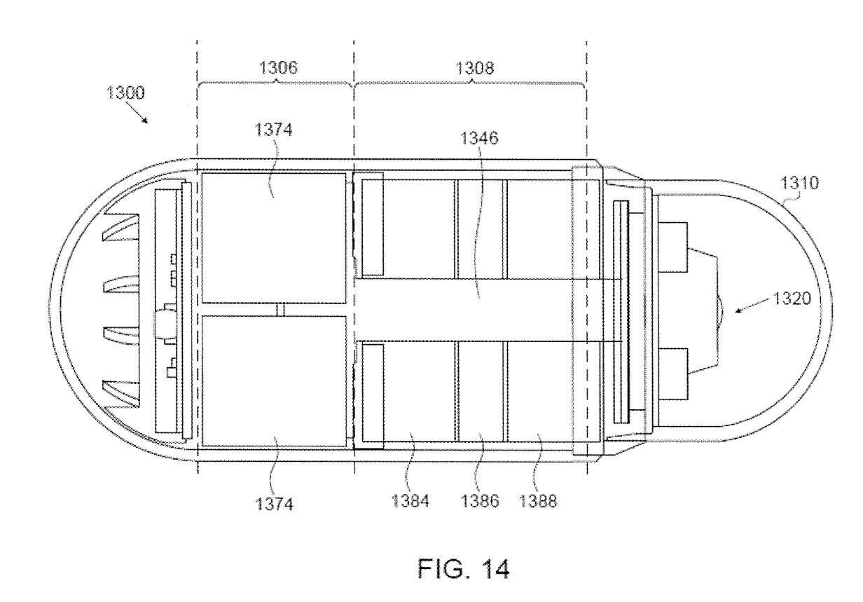

The examples described above (for example in connection with FIGS. 3C-3D and FIGS. 10A-10B) refer to a magnetic steering unit (MSU) in which the magnetic field sensing (MFS) section, when folded, and the permanent magnets assembly (PMA) fully structurally overlap cylindrically, annularly or concentrically. As explained above, an MSU may have other configurations in which the overlap between the MFS section, when folded, and the PMA is partial or non-existent. An example embodiment in which there is no structural overlap between the MFS section of the SCA and the PMA is shown in FIGS. 13A and 13B, and in FIG. 14, which are described bellow. Regarding FIGS. 13A-13B, FIG. 14 and FIGS. 15A-15B, like reference numerals refer to like elements, components, parts, or sections.

FIG. 13A and FIG. 13B show different cross-sectional views of an in-vivo device in which the MFS section of the SCA and the PMA do not overlap according to another example embodiment. According to this embodiment, the MFS section of the SCA and the PMA are located in different, non-overlapping, areas, or ‘sections’, of in-vivo device 1300, e.g., they are in non-overlapping areas/sections 1306 and 1308, respectively. The MFS section and the PMA may be adjacent to each other, as demonstrated by FIG. 13A (area/section 1306 and area/section 1308 are adjacent), and by FIGS. 13B and 14. In other embodiments, the MFS section and the PMA may be spaced apart (e.g., there may be a gap between them, e.g., 1-3 millimeters) with respect to a longitudinal axis 1302 of in-vivo device 1300.

As shown in FIG. 13A, in-vivo device 1300 may include a light transparent window 1310 which may be shaped, for example, as a dome; and an optical system 1320 that may include, for example, one or more lenses supported by a lens(es) holder. In-vivo device 1300 also includes a magnetic steering unit (MSU) to facilitate maneuvering of in-vivo device 1300.

The MSU may include a permanent magnets assembly (PMA) for steering in-vivo device 1300. The PMA may include a magnets carrying assembly (MCA) and one or more permanent magnets that may be held in, included in, or accommodated by the MCA. The MCA may be identical or similar to, and it may function in the same or similar manner as, for example, MCA 700 of FIG. 7D. By way of example, the MCA of in-vivo device 1300 includes a conductive tubular object 1390 and four annular conductive discs 1392, 1394, 1396, and 1396, that are disposed on the peripheral surface of conductive tubular object 1390.

Tubular object 1390 and four annular conductive discs 1392, 1394, 1396, and 1396 circumferentially form three open annular channels on the periphery of conductive tubular object 1390. The three open annular channels formed by the example conducting tubular object and the example four annular conductive discs are shown accommodating permanent annular magnets 1384, 1386, and 1386. The number of annular open channels may be three, less than three, or more than three, and the number of annular conductive discs may change accordingly. An annular open channel may include one or more permanent magnet(s), and the width of the annular open channel may change accordingly. By way of example, each annular open channel in FIG. 13A includes one permanent magnet. The annular conductive discs 1392, 1394, 1396, and 1398 in FIG. 13A are mutually parallel; in other embodiments the annular conductive discs may be unparallel.

In-vivo device 1300 may also include a multilayered imaging and sensing PCB (MISP) for sensing electromagnetic fields by which current location and/or current orientation and/or current angular position of the in-vivo device may be determined. The MISP may include, among other things, an SCA, for sensing electromagnetic fields, and a transmitter for transmitting data, which may correspond, for example, to or represent one or more sensed electromagnetic fields, to an external data recorder or maneuvering system. Turning back to FIG. 13A, the MISP may include a PCB section 1330, a PCB section 1340, a PCB section 1350, a PCB section 1360, a PCB section 1370, a PCB section 1372, and a magnetic field sensing (MFS) section 1374. A section of PCB sections 1330, 1340, 1350, 1360, 1370, and 1372 may be rigid or flexible. PCB section 1372 and MFS section 1374 may form the SCA part of the MISP.

Rigid PCB sections, for example rigid PCB sections of the MISP, may be structurally and electrically interconnected by one or more flexible PCB sections. A PCB section may be multilayered, where layers thereof may be electrically interconnected through vias. The entire, part, or most of the MISP may be flexible, while the other sections or parts of the MISP may be rigid. Electrical components (e.g., image sensor(s), ASIC, transmitter, illumination sources, controller, etc.) may be mounted on various PCB sections of the MISP. For example, illumination sources 1332 and 1334 are mounted on PCB section 1330 of the MISP; an image sensor 1342 and ASIC 1344 are mounted on PCB section 1340 of the MISP, a radio frequency (“RF”) operated switch 1352 and a conductive spring coil 1354 are mounted on PCB section 1350 of the MISP; various electrical components are generally shown, at 1362, mounted on PCB section 1360 of the MISP; additional electrical components (e.g., a controller 1376) are generally shown mounted on PCB section 1370 of the MISP.

MFS section 1374 may include (for example it may have mounted thereon, or embedded in, incorporated or formed therein) a set of electromagnetic sensing coils. PCB section 1372 may also include (for example it may have mounted thereon, or embedded in, incorporated or formed therein) an electromagnetic sensing coil that may functionally be part, or an extension, of MFS section 1374. Signals that are induced in the electromagnetic sensing coils of MFS section 1374 and PCB section 1372 by timely generated/transmitted sensing electromagnetic fields facilitate determination of the current location and/or current orientation and/or current angular position of the in-vivo device. Such determination may be made internally, for example, by controller 1376 of in-vivo device 1300 and communicated to an external system, or externally, for example by transmitting, from the in-vivo device to an external system, data that may represent the sensing coils' output in order for the external system to deduce the in-vivo device's current location and/or orientation and/or angular position from that data.

Magnetic field sensing (MFS) section 1374 is shown folded in FIGS. 13A-13B, and 14. Folded MFS section 1374 and housing 1304 of in-vivo device 1300 may make up concentric cylinders such that a longitudinal axis of MFS section 1374 and longitudinal axis 1302 of in-vivo device 1300 may be aligned; in other embodiments the two longitudinal axes may be misaligned. MFS section 1374 may include sensing coils whose setup may be identical or similar to the sensing coils' setup shown, for example, in FIG. 3A and described, for example, in connection with MFS 350.

In-vivo device 1300 also includes a power source that may include one or more batteries. By way of example, the power source of in-vivo device 1300 may include two batteries: battery 1380 and battery 1382. Batteries 1380 and 1382 may be rechargeable, for example they may be recharged by harvesting energy wirelessly; e.g., by exploiting electromagnetic radiation. Battery 1380 may be held in place between battery 1382 and PCB section 1350 by conductive spring coil 1354.

The length, L, of in-vivo device 1300 may be, for example, about 36 millimeters (e.g., 36.3 millimeters); the diameter, D, of in-vivo device 1300 may be, for example, about 13 millimeters (e.g., 13.4 millimeters). In-vivo device 1300 may have other lengths (e.g., 33 millimeters) and other diameters (e.g., 12 millimeters). Reference numeral 1378 designates a flexible PCB section of the in-vivo device's MISP that connects PCB section 1370 to PCB section 1372.

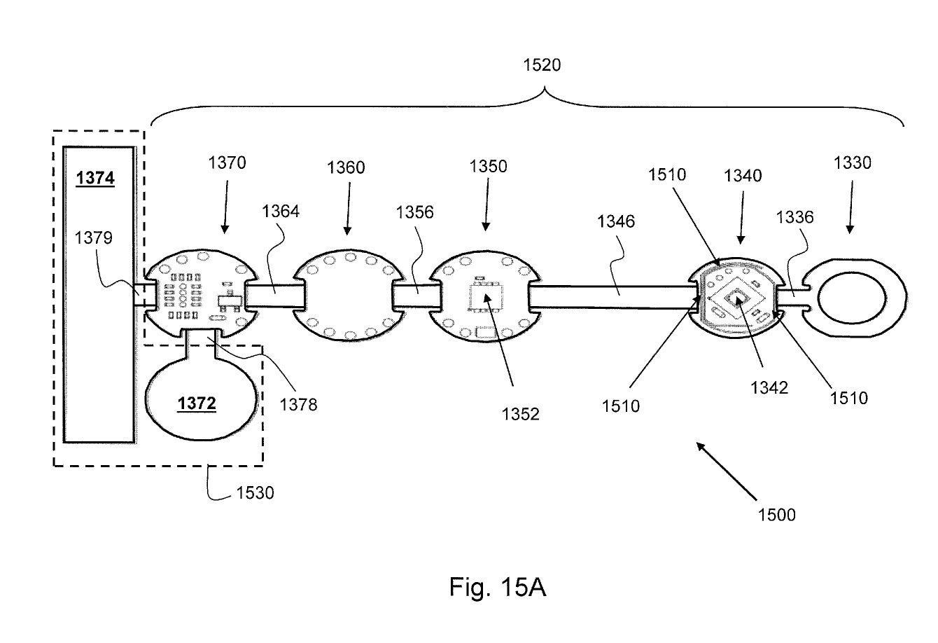

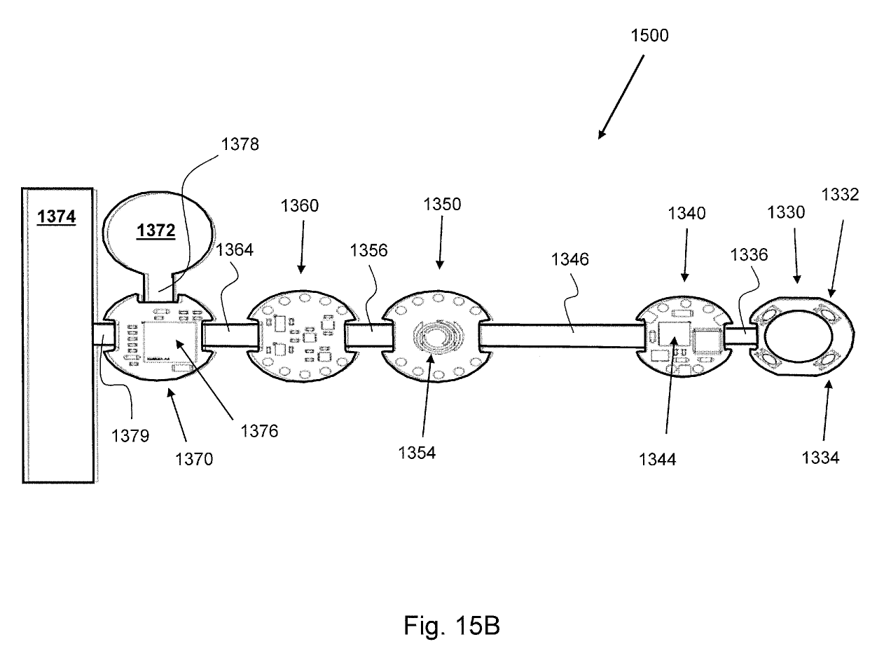

FIG. 13B shows another cross-sectional view of in-vivo device 1300. The MISP of in-vivo device 1300 may include PCB sections 1330, 1340, 1350, 1360, 1370, 1372, and 1374, and flexible PCB sections that connect these PCB sections. For example, flexible PCB section 1336 connects PCB sections 1330 and 1340; flexible PCB section 1346 connects PCB sections 1340 and 1350; flexible PCB section 1356 connects PCB sections 1350 and 1360; flexible PCB section 1364 connects PCB sections 1360 and 1370; flexible PCB section 1378 (shown in FIG. 13A) connects PCB sections 1370 and 1372; and flexible PCB section 1379 connects PCB sections 1370 and 1374. The MISP of the in-vivo device is shown folded in FIGS. 13A-13B, and 14, and spread out in FIGS. 15A and 15B.

FIG. 14 shows a general view of the in-vivo device of FIGS. 13A-13B. As can be seen in FIG. 14, there is no overlapping between MFS section 1374 and the PMA, as each section/part is located in a different area of in-vivo device 1300: MFS section 1374 in area 1306 and the PMA in area 1308.

FIG. 15A and FIG. 15B show two perspectives of a spread out multilayered imaging and sensing PCB (MISP) 1500 of in-vivo device 1300. In addition to the PCB sections and electrical components and circuitries mentioned above in connection with FIGS. 13A-13B, MISP 1500 may also include an antenna 1510 for transmitting, for example, images that are captured by, for example, image sensor 1342, and/or another type of data. The other type of data may be, or include, data pertaining to sensed electromagnetic fields that are used to determine the location and/or orientation and/or angular position of in-vivo device 1300. Antenna 1510 may be a coil including, for example, 1.5 turns, and it may be embedded in PCB section 1340, as shown in FIG. 15A. Referring to FIG. 15B, PCB section 1330 includes illumination sources 1332 and 1334 (e.g., LEDs), and it may include additional illumination sources.

MISP 1500 includes a primary PCB section 1520. Primary PCB section 1520 may include PCB sections 1330, 1340, 1350, 1360, and 1370, and the PCB sections that connect them. PCB sections 1330, 1340, 1350, 1360, and 1370 are lined up side by side, in a row. PCB section 1330, which may include the illumination source(s) (as shown in FIG. 15B, for example at 1332 and 1334), may be regarded as a first/leading PCB section of the PCB sections line up, and PCB section 1370 may be regarded as a second/trailing PCB section of the PCB sections line up. MISP 1500 also includes PCB section 1372.

MSF section 1374 may hold, include, or accommodate X-Y sensing coils (the sensing coils are not shown in FIGS. 15A-15B), for respectively sensing electromagnetic fields in the X axis and in the Y axis. PCB portion 1372 may hold, include, or accommodate a Z-axis sensing coil (the sensing coil is not shown in FIGS. 15A-15B), for sensing an electromagnetic field in the Z axis, where the Z axis may coincide with the longitudinal axis of the in-vivo device.

MFS section 1374 and PCB section 1372 make up, or form, SCA 1530. Trailing PCB section 1370, which is structurally and functionally connected to MFS section 1374 and to PCB section 1372 (via PCB section 1379 and PCB section 1378, respectively), may be regarded as a structural and functional PCB junction, or an intersection hub, that interconnects primary PCB section 1520 and SCA 1530.

In accordance with FIGS. 15A-15B, there is provided an embodiment in which a foldable multilayered imaging and sensing printed circuit board (MISP) for an in-vivo device may include a primary printed circuit board (PCB) section (e.g., primary PCB section 1520), the primary PCB section may include a first/leading PCB section (e.g., leading PCB section 1330), a second/trailing PCB section (e.g., trailing PCB section 1370), and one or more primary PCB sections that are disposed in-between the first/leading PCB section and the second/trailing PCB section (e.g., primary PCB sections 1340, 1350, and 1360). The first/leading PCB section, second/trailing PCB section and the one or more primary PCB sections may be interconnected (e.g., via PCB sections 1346, 1346, 1356, and 1364).

The MSIP may further include a sensing coils assembly (SCA) that may include a magnetic field sensing (MFS) section (e.g., MSF section 1374) and a PCB section (e.g., second PCB section 1372), the MFS section and the second PCB section may be connected via, or to, the (junction-like) second/trailing PCB section. The MSF section may include sensing coils for sensing electromagnetic fields in two axes of the X-Y-Z coordinates system (e.g., X and Y axes), and the PCB section/portion may include a sensing coil for sensing an electromagnetic field in a third axis (e.g., Z axis). The sensing coil that senses the electromagnetic field in the third axis and the PCB portion on which it is mounted or formed may be regarded as part of the MSF section.

The articles “a” and “an” are used herein to refer to one or to more than one (i.e., to at least one) of the grammatical object of the article, depending on the context. By way of example, depending on the context, “an element” can mean one element or more than one element. The term “including” is used herein to mean, and is used interchangeably with, the phrase “including but not limited to”. The terms “or” and “and” are used herein to mean, and are used interchangeably with, the term “and/or,” unless context clearly indicates otherwise. The term “such as” is used herein to mean, and is used interchangeably, with the phrase “such as but not limited to”.

Having thus described exemplary embodiments of the invention, it will be apparent to those skilled in the art that modifications of the disclosed embodiments will be within the scope of the invention. Alternative embodiments may, accordingly, include more modules, fewer modules and/or functionally equivalent modules. The present disclosure is relevant to various types of in-vivo devices (e.g., in-vivo devices with one or more imagers, in-vivo devices with no imagers at all, etc.), and to various types of electromagnetic field sensors (e.g., various types of magnetometers). Hence the scope of the claims that follow is not limited by the disclosure herein.

1. A swallowable in-vivo device comprising:
a foldable multilayered printed circuit board comprising:
an imaging section comprising an imaging circuit; and
an electromagnetic field sensing section comprising a first printed circuit board portion, said first printed circuit board portion comprising a first electromagnetic field sensing coil and a second electromagnetic field sensing coil,
wherein each layer of the multilayered printed circuit board comprises coil turns of the first electromagnetic field sensing coil and coil turns of the second electromagnetic field sensing coil, and wherein folding the electromagnetic field sensing section into a cylinder places some coil turns of the first electromagnetic field sensing coil opposite to other coil turns of the first electromagnetic field sensing coil to facilitate sensing of an electromagnetic field in a first direction, and some coil turns of the second electromagnetic field sensing coil opposite to other coil turns of the second electromagnetic field sensing coil to facilitate sensing of an electromagnetic field in a second direction different than the first direction.

2. The in-vivo device as in claim 1, wherein the multilayered printed circuit board comprises:
a primary printed circuit board branch, said primary printed circuit board branch comprises the imager;
a first secondary printed circuit board branch and a second secondary printed circuit board branch respectively intersecting the primary printed circuit board at a first intersection point and at a second intersection point; and
a tertiary printed circuit board branch intersecting the second secondary printed circuit board branch at a third printed circuit board intersection point, said tertiary printed circuit board branch comprising the electromagnetic field sensing section, and wherein the electromagnetic field sensing section further comprises a second printed circuit board portion comprising a third electromagnetic field sensing coil to facilitate sensing of an electromagnetic field in a third direction different than the first and second directions.

3. The in-vivo device as in claim 2, wherein the first secondary printed circuit board branch comprises a light emitting diode ring.

4. The in-vivo device as in claim 2, wherein the first secondary printed circuit board branch comprises a first battery contact and the second secondary printed circuit board branch comprises a second battery contact.

5. The in-vivo device as in claim 2, wherein each printed circuit board intersection point is a printed circuit board portion common to the intersecting printed circuit board branches.

6. The in-vivo device as in claim 2, wherein portions of the printed circuit board include four printed circuit board layers.

7. The in-vivo device as in claim 2, wherein the first printed circuit board portion of the electromagnetic field sensing section comprises X-Y sensing coils for respectively sensing electromagnetic field components in the X-direction and Y-direction, and wherein the second printed circuit board portion of the electromagnetic field sensing section comprises a Z sensing coil for sensing an electromagnetic field in the Z-direction.

8. The in-vivo device as in claim 2, wherein the primary printed circuit board branch and the secondary printed circuit board branches are foldable such that, after folding, some portions of the primary printed circuit board branch and portions of the secondary printed circuit board branches are stacked in parallel and the other portions connect the stacked parallel portions.

9. The in-vivo device as in claim 2, wherein the tertiary printed circuit board branch is fully flexible.

10. The in-vivo device as in claim 1, wherein the printed circuit board includes rigid portions and flexible portions.

11. The in-vivo device as in claim 1, wherein the printed circuit board is fully flexible.

12. The in-vivo device as in claim 1, wherein the multilayered printed circuit board comprises:
a primary printed circuit board section, said primary printed circuit board section comprising a first printed circuit board section, a second printed circuit board section, and one or more printed circuit board sections interposed between the first printed circuit board section and the second printed circuit board section, the first printed circuit board section, second printed circuit board section, and the one or more printed circuit board sections being interconnected via flexible printed circuit board sections,
wherein the electromagnetic field sensing section further comprises a second printed circuit board portion comprising a third electromagnetic field sensing coil to facilitate sensing of an electromagnetic field in a third direction, and wherein the first printed circuit board portion and the second printed circuit board portion of the electromagnetic field sensing section are connected to the second printed circuit board section.

13. The in-vivo device as in claim 1, further comprising a magnetic steering unit maneuverable by an external electromagnetic field, said magnetic steering unit comprising:
a permanent magnets assembly for interacting with the electromagnetic field to produce a propelling force and a rotational force for moving and rotating the in-vivo device, said permanent magnets assembly comprising at least one permanent magnet; and
a magnet carrying assembly to accommodate the at least one permanent magnet, said magnet carrying assembly configured to interact with an electromagnetic field to generate eddy currents to produce a repelling force.

14. The in-vivo device as in claim 13, wherein the permanent magnets assembly and the sensing coil assembly partly or fully structurally and concentrically overlap.

15. The in-vivo device as in claim 13, wherein the permanent magnets assembly and the sensing coil assembly do not structurally and concentrically overlap.
